SEQUENCE LISTING (36 sequences)

SEQ ID NO: 1 - DNA, 20 bp, Artificial Sequence
  Synthetic Polynucleotide
ttcgctgtctaccttccagc

SEQ ID NO: 2 - DNA, 20 bp, Artificial Sequence
  Synthetic Polynucleotide
aagcgcaagaaatcccggtt

SEQ ID NO: 3 - DNA, 23 bp, Artificial Sequence
  Synthetic Polynucleotide
tctactgggatcgagacatgtga

SEQ ID NO: 4 - DNA, 20 bp, Artificial Sequence
  Synthetic Polynucleotide
agcttggaacggtacgctaa

SEQ ID NO: 5 - DNA, 21 bp, Artificial Sequence
  Synthetic Polynucleotide
actgccaagaaaatggtcacg

SEQ ID NO: 6 - DNA, 20 bp, Artificial Sequence
  Synthetic Polynucleotide
acgtggaaacccgaggtatg

SEQ ID NO: 7 - DNA, 24 bp, Artificial Sequence
  Synthetic Polynucleotide
agattggagtgtgcactgccaaag

SEQ ID NO: 8 - DNA, 23 bp, Artificial Sequence
  Synthetic Polynucleotide
cccaactggtacatcagcacctc

SEQ ID NO: 9 - DNA, 21 bp, Artificial Sequence
  Synthetic Polynucleotide
tgccaccttttgacagtgatg

SEQ ID NO: 10 - DNA, 23 bp, Artificial Sequence
  Synthetic Polynucleotide
tagtccttcctaccccaatttcc

SEQ ID NO: 11 - DNA, 20 bp, Artificial Sequence
  Synthetic Polynucleotide
tccagcatttctgctccaca

SEQ ID NO: 12 - DNA, 21 bp, Artificial Sequence
  Synthetic Polynucleotide
gagctgtttgcagacaaagtc

SEQ ID NO: 13 - DNA, 23 bp, Artificial Sequence
  Synthetic Polynucleotide
agtactgccgaggcctgtaataa

SEQ ID NO: 14 - DNA, 21 bp, Artificial Sequence
  Synthetic Polynucleotide
tctggaattgtaccgcagctt

SEQ ID NO: 15 - DNA, 20 bp, Artificial Sequence
  Synthetic Polynucleotide
agggctaccatgccaacttc

SEQ ID NO: 16 - DNA, 21 bp, Artificial Sequence
  Synthetic Polynucleotide
cgagaccaccttataccagcg

SEQ ID NO: 17 - DNA, 20 bp, Artificial Sequence
  Synthetic Polynucleotide
ccctcacactcacaaaccac

SEQ ID NO: 18 - DNA, 20 bp, Artificial Sequence
  Synthetic Polynucleotide
gtagcccacgtcgtagcaaa

SEQ ID NO: 19 - DNA, 20 bp, Artificial Sequence
  Synthetic Polynucleotide
gaggcgctgatccacaaaac

SEQ ID NO: 20 - DNA, 22 bp, Artificial Sequence
  Synthetic Polynucleotide
gcttgtcacatctgcaagtacg

SEQ ID NO: 21 - DNA, 24 bp, Artificial Sequence
  Synthetic Polynucleotide
attgctatttctttctgcgtgcat

SEQ ID NO: 22 - DNA, 20 bp, Artificial Sequence
  Synthetic Polynucleotide
gagtccactgacttgctccc

SEQ ID NO: 23 - DNA, 20 bp, Artificial Sequence
  Synthetic Polynucleotide
caatggacacggagcatcca

SEQ ID NO: 24 - DNA, 20 bp, Artificial Sequence
  Synthetic Polynucleotide
ttgggtccctcgactcctac

SEQ ID NO: 25 - DNA, 24 bp, Artificial Sequence
  Synthetic Polynucleotide
tccaggcaagtgcattggtatttg

SEQ ID NO: 26 - DNA, 23 bp, Artificial Sequence
  Synthetic Polynucleotide
gacacggattccatggtgaagtc

SEQ ID NO: 27 - DNA, 20 bp, Artificial Sequence
  Synthetic Polynucleotide
tgatgtgctgctgcgagatt

SEQ ID NO: 28 - DNA, 21 bp, Artificial Sequence
  Synthetic Polynucleotide
ttggtccttagccactccttc

SEQ ID NO: 29 - DNA, 20 bp, Artificial Sequence
  Synthetic Polynucleotide
acagactcggcactcaatgg

SEQ ID NO: 30 - DNA, 20 bp, Artificial Sequence
  Synthetic Polynucleotide
ccctggcacatgaatcctgg

SEQ ID NO: 31 - DNA, 19 bp, Artificial Sequence
  Synthetic Polynucleotide
ttgttcctcaggccccaat

SEQ ID NO: 32 - DNA, 21 bp, Artificial Sequence
  Synthetic Polynucleotide
atgactgcagcaaatcgcttg

SEQ ID NO: 33 - DNA, 19 bp, Artificial Sequence
  Synthetic Polynucleotide
ccacgtagtagacgatggc

SEQ ID NO: 34 - DNA, 20 bp, Artificial Sequence
  Synthetic Polynucleotide
atgactggggtgtaggcgta

SEQ ID NO: 35 - DNA, 20 bp, Artificial Sequence
  Synthetic Polynucleotide
atagcaaatcggctgacggt

SEQ ID NO: 36 - DNA, 20 bp, Artificial Sequence
  Synthetic Polynucleotide
acaaggtacaacccatcggc

(19) United States
(12) Patent Application Publication
(10) Pub. No.: US 2022/0241339 A1
(43) Pub. Date: Aug. 4, 2022

(54) ABCB5+ STEM CELL THERAPEUTICS FOR LIVER DISEASE

(71) Applicants: Children's Medical Center Corporation, Boston, MA (US); TICEBA GmbH, Heidelberg (DE)

(72) Inventors: Markus H. Frank, Cambridge, MA (US); Christoph Ganss, Heidelberg (DE); Mark Andreas Kiuth, Heidelberg (DE)

(21) Appl. No.: 17/605,862

(22) PCT Filed: Apr. 24, 2020
(86) PCT No.: PCT/US2020/029833

§ 371 Date: Oct. 22, 2021

Related U.S. Application Data
(60) Provisional application No. 62/838,062, filed on Apr. 24, 2019.

(51) Int. Cl. A61K 35/28; A61P 1/16

(52) U.S. Cl. CPC A61K 35/28; A61K 9/0019; A61P 1/16

(57) ABSTRACT

The invention relates to methods for treating liver disease. For instance a method for treating liver fibrosis in a subject in a subject in need thereof, comprising injecting an isolated population of ABCB5+ stem cells into the subject in an effective amount to treat liver fibrosis in the subject is provided. Methods for inducing liver tissue generation in a subject in need thereof by injecting an isolated population of ABCB5+ stem cells into the subject in an effective amount to generate liver tissue are also provided.

### RELATED APPLICATIONS

This application claims priority under 35 U.S.C. § 119(e) to U.S. Provisional Application Ser. No. 62/838,062, filed on Apr. 24, 2019 which is herein incorporated by reference in its entirety.

### BACKGROUND OF INVENTION

Liver transplantation is still the main effective treatment for patients with end-stage liver disease, hepatocellular carcinoma (HCC), and acute liver failure [1-3]. However, this option has several limitations including high cost, shortage of donor pool, the side effects of immunosuppressive therapy, long-term outcomes, and recurrent disease in the transplanted liver [3]. In Europe for example, patients on the waiting list are twice the number of available liver organs for transplantation [4]. Therefore, new therapeutic alternatives such as cell-based therapy are highly encouraged [5]. For instance, primary hepatocytes (PHH) transplantation improved liver function parameters in patients with inherited metabolic liver diseases [6, 7] and acute liver failure [8]. Unfortunately, the usage of PHH is hampered by the limited proliferation of PHH and the difficulty obtaining a healthy liver organ for cell isolation on routine basis [8, 9].

### SUMMARY OF THE INVENTION

In some aspects the invention is a method for inducing liver tissue generation in a subject in need thereof, by injecting an isolated population of ABCB5+ stem cells into the subject in an effective amount to generate liver tissue.

In other aspects the invention is a method for treating liver fibrosis in a subject in need thereof, by injecting an isolated population of ABCB5+ stem cells into the subject in an effective amount to treat liver fibrosis in the subject.

In some embodiments the ABCB5+ stem cells are cultured in a hepatocytic differentiation medium prior to injection into the subject.

In other embodiments the ABCB5+ stem cells are injected into the liver of the subject. In yet other embodiments the ABCB5+ stem cells are injected systemically into the subject.

In some embodiments the ABCB5+ stem cells are ABCB5+ dermal stem cells. In other embodiments the ABCB5+ stem cells are ABCB5+ limbal stem cells. In yet other embodiments, the ABCB5+ stem cells are synthetic ABCB5+ stem cells.

The ABCB5+ stem cells may be ABCB5+ dermal mesenchymal stem cells. In some embodiments at least 85% or 90% of the population of stem cells are ABCB5+ stem cells.

In other embodiments of the invention, ABCB5(+) stem cells are limbal or retinal stem cells. ABCB5(+) stem cells may be obtained from (e.g., isolated from or derived from) the basal limbal epithelium of the eye or from the retinal pigment epithelium (RPE). In some embodiments, ABCB5(+) stem cells are obtained from human eye. Other ABCB5(+) stem cell types such as, for example, those obtained from the central cornea may be used in various aspects and embodiments of the invention.

In some embodiments the ABCB5+ stem cells for use in the invention are a population of synthetic ABCB5+ stem cells. Greater than 96% of the population is an in vitro progeny of physiologically occurring skin-derived ABCB5-positive mesenchymal stem cells is provided. In some embodiments greater than 96.5%, 97%, 97.5%, 98%, 98.5%, 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells. In some embodiments, 100% of the population is an in vitro progeny of physiologically occurring skin-derived ABCB5-positive mesenchymal stem cells.

In some embodiments greater than 90% of the synthetic stem cells in the population co-express CD90. In other embodiments the population of synthetic stem cells are capable of VEGF secretion under hypoxia as measured by ELISA. In other embodiments the population of synthetic stem cells are capable of IL-1RA secretion after co-culture with Mi-polarized macrophages. In other embodiments the population of synthetic stem cells induce decreased TNF-alpha and IL-12/IL-23p40 secretion, and increased IL-10 secretion, in macrophage co-culture relative to isolated physiologically occurring skin-derived ABCB5-positive mesenchymal stem cells. In other embodiments the population of synthetic stem cells possess multipotent differentiation capacity. In other embodiments the population of synthetic stem cells possess the capacity to differentiate into cells derived from all three germ layers, endoderm, mesoderm and ectoderm. In other embodiments the population of synthetic stem cells possess corneal epithelial differentiation capacity. In other embodiments the population of synthetic stem cells exhibit increased expression of stem cell markers including SOX2, NANOG and SOX3 relative to isolated physiologically occurring skin-derived ABCB5-positive mesenchymal stem cells. In other embodiments the population of synthetic stem cells exhibit decreased expression of mesenchymal stromal differentiation markers including MCAM, CRIG1 and ATXN1 relative to isolated physiologically occurring skin-derived ABCB5-positive mesenchymal stem cells. In other embodiments at least 5% of the population of synthetic stem cells includes an exogenous gene. In other embodiments at least 10%, 15%, 20%, 25%, 30%, 35%, 40%, 45%, 50%, 55%, 60%, 65%, 70%, 75%, 80%, 85%, 90%, or 95% of the population of synthetic stem cells includes an exogenous gene. In other embodiments the exogenous gene is a gene encoding a protein selected from the group consisting of tissue-specific homing factors, secreted tissue remodeling proteins, growth factors, cytokines, hormones and neurotransmitters. In other embodiments at least 5% of the population of synthetic stem cells comprise a modification in a gene. In other embodiments at least 10%, 15%, 20%, 25%, 30%, 35%, 40%, 45%, 50%, 55%, 60%, 65%, 70%, 75%, 80%, 85%, 90%, or 95% of the population of synthetic stem cells comprise a modification in a gene.

Use of a population of stem cells of the invention for treating liver fibrosis disorders as described herein or liver tissue engineering is also provided as an aspect of the invention.

A method for manufacturing a medicament of a population of stem cells of the invention for treating the disorders as described herein or tissue engineering is also provided.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C: Treatment conditions and safety profile of ABCB5⁺ cell therapy. (FIG. 1A) Treatment scheme. Values for (FIG. 1B) body weight, liver/body weight ratio, and (FIG. 1C) liver function parameters of both immunosuppressed (is) and non-immunosuppressed (nis) Mdr2KO mice, treated or not with ABCB5⁺ cells (n=8 for 4 weeks, n=4 for 2 days and 2 weeks). Liver tissues were examined at the indicated time points. Black dots represent is saline-treated Mdr2KO mice, red dots indicate ABCB5⁺ cell-treated Mdr2KO mice. Triangles represent nis and squares is ABCB5⁺ mice with double cell treatment. ALT, alanine aminotransferase; AST, aspartate aminotransferase; TG, triglyceride; AP, alkaline phosphatase.

FIGS. 2A-2B: Effects of ABCB5⁺ cells treatment on biliary fibrosis in Mdr2KO mice. Representative histochemical/IHC staining for (FIG. 2A) Sirius red and (FIG. 2B) αSMA in 10 and 40 fold magnification. Quantification of positive staining is presented as scatter plots, discriminating periportal, pericentral and midzonal areas, as indicated for n=4-8 for 4 weeks, n=4 for 6 days and 2 weeks' time points. Black dots represent is saline-treated Mdr2KO mice, red dots indicate ABCB5⁺ cell-treated Mdr2KO mice. Triangles represent nis and squares is ABCB5⁺ mice with double cell treatment.

FIGS. 3A-3C: Effects of ABCB5⁺ cells treatment on liver inflammation and proliferation in Mdr2KO mice. IHC staining of (FIG. 3A) F4/80 and (FIG. 3B) CD163 in 10 and 40 fold magnification. Quantification of positive staining is presented as scatter plots, discriminating periportal, pericentral and midzonal areas, as indicated for n=4-8 for 4 weeks and n=4 for 6 days and 2 weeks' time points (FIG. 3C) Real time RT-PCR data for IL-1γ and IL-10, as indicated. Black dots represent is saline-treated Mdr2KO mice, red dots indicate ABCB5⁺ cell-treated Mdr2KO mice. Triangles represent nis and squares is ABCB5⁺ mice with double cell treatment.

FIG. 4: Effects of ABCB5⁺ cells treatment on cell proliferation in Mdr2KO mice. IHC staining of Ki-67 positive staining in 10 and 40 fold magnification. Quantification of positive staining is presented as scatter plot, discriminating periportal, pericentral and midzonal areas, as indicated for n=4 at 2 weeks' time point. Black dots represent is saline-treated Mdr2KO mice, red dots indicate ABCB5⁺ cell-treated Mdr2KO mice.

FIGS. 5A-5B: Effects of ABCB5⁺ cells-generated secretome on LX-2 cells. LX-2 cells were treated with supernatant of ABCB5⁺ cells kept under different conditions as indicated. (FIG. 5A) Representative immunoblot with antibodies as indicated and quantification of the respective signal intensities from 3 independent replicas, using ImageJ software. (FIG. 5B) Real time RT-PCR data for selected fibrogenesis related gene expression. P, supernatant of cultured ABCB5⁺ cells from different patients. MΦ, supernatant of activated macrophages MΦ+, supernatant of ABCB5⁺ cells stimulated by co-culturing with activated macrophages. S+, supernatant of cultured ABCB5⁺ cells stimulated with INF-g. LX-2 cells treated with TGF-β1 or LY (LY2157299) are included as positive and negative controls of activated TGF-β signaling.

FIGS. 6A-6D: Assessment of fibrogenic markers. Collagen fibers were visualized with the Sirius red stain (FIG. 6A) 2 days and 7 weeks after transplantation of ABCB5⁺ cells into livers of Pfp/Rag2KO mice. HRG was used as carrier control. Pictures show representative images of slices from 2 animals in each group. Scale bar—100 μm. Using semiquantitative RT-PCR (FIG. 6B), the relative expression of mRNAs of TIMP1 and VEGF was determined 2 days (white columns) and 7 weeks (grey columns) after transplantation of ABCB5⁺ cells. Values shown are means±SD of n=4 animals in each group (*p<0.05). Housekeeping genes β2-microglobulin and TATA-box binding protein were used for normalization 903 of expression levels. (FIG. 6C, FIG. 6D) Relative expression of inflammation (FIG. 6C) and apoptosis (FIG. 6D) markers in livers of Pfp/Rag2KO mice 2 days (white columns) and 7 weeks (grey columns) after transplantation of ABCB5⁺ cells. Values shown are means±SD of n=4 animals in each group (*p<0.05). Housekeeping genes β2-microglobulin and TATA-box binding protein were used for normalization of expression levels.

FIGS. 7A-7B: Comparison of male and female Mdr2KO mice. Quantification of Sirius red staining (FIG. 7A) in control mice at different ages, as indicated, and (FIG. 7B) after ABCB5⁺ cells treatment for the indicated time points; positive staining is presented as scatter plots, discriminating periportal, pericentral and midzonal areas, as indicated for n=4-8 for 4 weeks, n=4 for 6 days and 2 weeks time points. Black dots represent is saline-treated Mdr2KO mice, red dots indicate ABCB5⁺ cell-treated Mdr2KO mice. Triangles represent nis and squares is ABCB5⁺ mice with double cell treatment. Pink dots are female mice; nt, not treated; t, treated with ABCB5⁺ cells.

FIGS. 8A-8C: Effects of ABCB5⁺ cells treatment on biliary fibrosis in Mdr2KO mice. Quantification of (FIG. 8A) αSMA and (FIG. 8B) SMA22α positive staining is presented as scatter plots, discriminating periportal, pericentral and midzonal areas, as indicated for n=4-8 for 4 weeks, n=4 for 6 days and 2 weeks time points. Representative IHC staining for SMA22α is shown in 10 and 40 fold magnification. (FIG. 8C) Real time RT-PCR data for selected fibrogenesis related gene expression, as indicated, and biochemical determination of hydroxyproline levels (HYP). Black dots represent is saline-treated Mdr2KO mice, red dots indicate ABCB5⁺ cell-treated Mdr2KO mice. Triangles represent nis and squares is ABCB5⁺ mice with double cell treatment.

FIGS. 9A-9C: Effects of ABCB5⁺ cell treatment on liver inflammation in Mdr2KO mice. (FIG. 9A) CD45 and (FIG. 9B) CD163 positive staining is presented as scatter plots, discriminating periportal, pericentral and midzonal areas, as indicated for n=4-8 for 929 4 weeks, n=4 for 2 days and 2 weeks' time points. IHC staining of CD45 (FIG. 9A) is presented in 10 and 40 fold magnification. Quantification of positive staining is presented as scatter plots, discriminating periportal, pericentral and midzonal areas, as indicated for n=4 for 6 days and 2 weeks' time points. Black dots represent is saline-treated Mdr2KO mice, brown dots indicate ABCB5⁺ cells-treated Mdr2KO mice. (FIG. 9C) Real time RT-PCR data for selected inflammation related gene expression, as indicated. Black dots represent is saline-treated Mdr2KO mice, red dots indicate ABCB5⁺ cell-treated Mdr2KO mice. Triangles represent nis and squares is ABCB5⁺ mice with double cell treatment.

FIGS. 10A-10B: Effects of ABCB5⁺ cells treatment on apoptosis and proliferation in Mdr2KO mice. (FIG. 10A) Representative documentation of apoptotic cells based on TUNEL assays and immunofluorescent staining (n=4) 2 and 6 days after cell treatment. (FIG. 10B) Quantification of Ki-67 immunostaining is presented as scatter plots, discriminating periportal, pericentral and midzonal areas, as indicated of n=8 for 4 weeks and n=4 for 6 days and 2 weeks' time points. Black dots represent is saline-treated Mdr2KO mice, red dots indicate ABCB5⁺ cell-treated Mdr2KO mice. Triangles represent nis and squares is ABCB5⁺ mice with double cell treatment.

### DESCRIPTION OF THE INVENTION

In previous work, it has been shown that ABCB5⁺ cells did not stimulate hepatocyte proliferation in a mouse model of liver regeneration after one third partial hepatectomy (Assessment of the hepatocytic differentiation ability of human skin-derived ABCB5⁺ stem cells. Tietze L, Winkler S, Hempel M, Kluth M A, Tappenbeck N, Ganss C, Dooley S, Christ B. Exp Cell Res. 2018 Aug. 15;369(2):335-347). Additionally, MSC from bone marrow have been suspected to have fibrogenic potential (The bone marrow functionally contributes to liver fibrosis. Russo F P, Alison M R, Bigger B W, Amofah E, Florou A, Amin F, Bou-Gharios G, Jeffery R, Iredale J P, Forbes S J. Gastroenterology. 2006 May;130(6):1807-21), thus possibly counteracting any potential liver fibrotic therapy. In contrast to these findings it was discovered that ABCB5+ stem cells had positive anti fibrotic and tissue regenerative properties when delivered to the liver in vivo. A mouse model of liver regeneration after partial hepatectomy was used, to test the activity of the transplanted ABCB5⁺ cells and is described in the Examples section.

The fact that the potential of ABCB5⁺ to transdifferentiate into the hepatocytic lineage had failed when tested was disappointing. However, the unexpected discoveries of the instant invention reveal that when administered in vivo ABCB5+ stem cells in fact are effective in addressing liver associated disease. As shown in the Examples, upon culture in hepatocytic differentiation medium, these cells produce and secrete a number of immunomodulatory, anti-fibrotic and hepatocyte fate modulating factors. Hepatic transplantation of such pretreated ABCB5⁺ stem cells was non-toxic and the liver architecture was maintained. It was further demonstrated that human ABCB5⁺ skin-derived stem cells secreting putative hepatotropic factors when injected into Mdr2KO mice have a therapeutic effect on liver fibrosis. Mice on the Balb/c background were selected for cell injections to investigate the effects on established fibrosis and inflammation.

Importantly, cell injections into mice upon acute damage by partial liver resection or fibrotic Mdr2KO mice did not result in obvious systemic or liver toxicity. ABCB5⁺ cell treatment consistently and significantly reduced the amount of collagen deposition, especially in the periportal region of the established biliary liver fibrosis in the Mdr2KO mice. Furthermore, ABCB5⁺ cells showed some influence on the liver inflammatory response. In line with these results, a tendency of reduced apoptosis and enhanced regenerative proliferation was evident. In vitro experiments with LX-2 human hepatic stellate cells display that the supernatant of ABCB5⁺ cells is shaping the phenotype of these cells, as was shown with induced TGF-β signalling, decreases in αSMA and upregulation of Vimentin and CTGF expression.

It was observed, unexpectedly, that collagen deposition was reduced in the periportal region following injection of ABCB5+ stem cells throughout the time period tested (6 days, 2 and 4 weeks). In contrast to this finding, expression of fibrosis marker genes, including SM22α, a marker of activated portal fibroblasts, were not downregulated. αSMA was even induced at the mRNA level at the 6 day time point. These findings suggested that the observed reduction of collagen deposition after stem cell therapy resulted from matrix degradation rather than a decrease in fibrogenesis. This may have been particularly relevant given that the time point of therapy coincided with already established fibrosis.

Based on the results of an in vitro study with LX-2 cells (incubated with the supernatant of cultured ABCB5⁺ cells) the TGF-β signalling pathway and several other selected marker genes play a role in the observed activity. R-Smad2/3 phosphorylation was induced to a similar extent in LX-2 cells treated with 5 ng TGF-β or cell supernatant, which suggests that either Activin or TGF-β are produced and secreted from the ABCB5⁺ cells. LX-2 cells are activated HSCs and TGF-β treatment does not further induce βSMA expression. However, ABCB5⁺ cell supernatant strongly enhanced αSMA expression. It was concluded that another factor other than TGF-β was responsible for this effect. Furthermore, Vimentin—another mesenchymal marker protein is strongly downregulated in LX-2 cells after ABCB5⁺ cell supernatant treatment.

Similar to the observations seen with the Mdr2KO mice, fibrogenesis related genes were not induced by treatment with ABCB5+ cell supernatant, with the exception of CTGF, which is induced similarly with TGF-β. CTGF is a secreted multifunctional protein, which is upregulated in several liver diseases including liver regeneration (Ujike et al., 2000), HCC (Hirasaki, Koide, Ujike, Shinji, & Tsuji, 2001), and liver fibrosis (Rachfal & Brigstock, 2003). Its profibrogenic activity is mainly due to its ability to induce proliferation, adhesion, migration and to increase ECM secretion of Myofibroblasts and HSCs (Rachfal & Brigstock, 2003). In cholestatic liver diseases, intense staining for CTGF has been shown around the portal area (Abshagen et al., 2015; Sedlaczek et al., 2001).

ABCB5⁺ cells demonstrate paracrine effects in non-healing wounds. Local injection of the cells accelerated wound healing in the iron overload mouse model that recapitulates the non-healing state of human venous ulcers. Beneficial effects have been dedicated to production of interleukin-1 receptor antagonist (IL-1RA) secretion and blunted inflammation with decreased IL-1β and TNFα release and a shift from proinflammatory M1 macrophages towards anti-inflammatory, repair promoting M2 macrophages.

In comparison to this study, where the cells were directly applied to the wound area, the study described in the examples used cell injection via the tail vain, far away from the target organ. Additionally, the effects were more indirectly examined, e.g. 2 and 4 weeks after the cell injection. Nevertheless, several effects on inflammatory cells were observed, which were different than those from the wound healing study. Macrophages have been functionally categorized into two major groups, M1 and M2, although it is now known that many more fates may exist (Liver macrophages in tissue homeostasis and disease. Krenkel O, Tacke F. Nat Rev Immunol. 2017 May;17(5):306-321. doi: 10.1038/nri.2017.11. Epub 2017 Mar. 20. Review). M1 macrophages are pro-inflammatory cells that secrete a plethora of pro-inflammatory cytokines and chemokines, whereas M2 macrophages have essential roles in tissues homeostasis and cellular repair (Ma, Yang, He, Wei, & Li, 2017). F4/80 is a general macrophage marker, while CD163, is a scavenger receptor, which marks M2 anti-inflammatory macrophages in rat and human (Fabriek, Dijkstra, & van den Berg, 2005, p. 163). The contribution of macrophages to liver diseases depends on their subtypes and the stage of diseases. For example, removal of macrophages during the development of CCl₄-mediated liver fibrosis was beneficial. On the other hand, deletion of macrophages during fibrosis resolution interfered with ECM degradation, which delayed tissue repair (Duffield et al., 2005). In PSC patients, M1 and M2 macrophages were increased around the biliary area and in an acute model of sclerosing cholangitis, macrophages were polarized to M1 subtype during the injury phase, while M2 were the main macrophages during the resolution process (Guicciardi et al., 2018). These results suggest a principal beneficial role of M2 macrophages in biliary fibrosis. In the present study, F4/80 positive macrophages displayed a dynamic response with decreased numbers at day 6 and increased positive staining at the 2 and 4 weeks time points. CD163 positive M2 macrophages were decreased in numbers in the midzones of the liver lobules as a result of ABCB5⁺ cell treatment, whereas the number of CD45 positive leukocytes did not vary at the time points investigated.

With regard to cytokines, IL-1β and IL-10 mRNA levels were upregulated at the 6 days time point upon ABCB5⁺ cell treatment. IL-1β is a proinflammatory cytokine secreted in an inactive form. Active IL-1β is generated through inflammasome complex containing caspase 1 (Tsutsui, Cai, & Hayashi, 2015). Mature IL-1β acts via IL1r1/2 to enhance the inflammatory reactions of several liver diseases, such as non-alcoholic steatohepatitis (Miura et al., 2010), paracetamol-mediated liver damage (Imaeda et al., 2009), liver fibrosis (Gieling, Wallace, & Han, 2009), and alcoholic steatohepatitis (Petrasek et al., 2012). In the wound healing approach, upregulation of IL-1RA by ABCB5⁺ cells was evident, which was not found in the livers, at least at mRNA levels.

With regard to liver damage and regeneration, there was a tendency of reduced cell death at 2 and 6 days after cell transplantation, whereas at the 2 weeks time point, proliferative activity of hepatocytes was significantly increased, suggesting a beneficial effect towards liver regeneration. The ability of ABCB5⁺ cells to facilitate differentiation of liver progenitor cells to functional hepatocytes is a significant advance, especially in patients with late stage liver diseases.

The cells useful according to the invention are ABCB5 positive stem cells. ABCB5 is a novel and important marker for the isolation of multipotent stem cell populations from normal human tissue. “ABCB5(+) stem cells,” as used herein, refers to cells having the capacity to self-renew and to differentiate into mature cells of multiple adult cell lineages. These cells are characterized by the expression of ABCB5 on the cell surface. In some embodiments of the invention, ABCB5(+) stem cells are dermal or ocular stem cells. In other embodiments the ABCB5(+) stem cells are synthetic stem cells.

“ABCB5 positive dermal mesenchymal stem cells” as used herein refers to cells of the skin having the capacity to self-renew and to differentiate into mature cells of multiple adult cell lineages such as bone, fat and cartilage. These cells are characterized by the expression of ABCB5 on the cell surface. In culture, mesenchymal stem cells may be guided to differentiate into bone, fat, cartilage, or muscle cells using specific media. (Hirschi K K and, Goodell M A. Gene Ther. 2002; 9: 648-652. Pittenger M F, et al., Science. 1999; 284: 143-147. Schwartz R E, et al., J Clin Invest. 2002; 109: 1291-1302. Hirschi K and Goodell M. Differentiation. 2001; 68: 186-192.)

The ABCB5 positive dermal mesenchymal stem cells can be obtained from skin. The skin may be derived from any subject having skin, but in some embodiments is preferably human skin. The skin may be derived from a subject of any age but in some embodiments is preferably adult skin, rather than adolescent or infant skin.

ABCB5⁺ cells have been identified as a phenotypically distinct dermal cell population able to provide immunregulatory functions. Greater than 90% of ABCB5⁺ cells express MSC markers CD29, CD44, CD49e, CD73, CD105, and CD166, as well as the immune checkpoint receptor PD-1.

In other embodiments of the invention, ABCB5(+) stem cells are ocular stem cells. ABCB5(+) stem cells may be obtained from (e.g., isolated from or derived from) the basal limbal epithelium of the eye or from the retinal pigment epithelium (RPE). In some embodiments, ABCB5(+) stem cells are obtained from human eye. Other ABCB5(+) stem cell types such as, for example, those obtained from the central cornea may be used in various aspects and embodiments of the invention.

The cells of the invention also may possess multipotent differentiation capacity. In other words these cells not only define mesenchymal stromal cells (adipogenic, chondrogenic, osteogenic differentiation), but also other capacities, including differentiation to cells derived from of all three germ layers, i.e. 1. endoderm (e.g. angiogenesis—e.g. tube formation, CD31 and VEGFR1 expression), 2. mesoderm (e.g. myogenesis—e.g. spectrin, desmin expression) and 3. ectoderm (e.g. neurogenesis—e.g. Tuj1 expression).

In other embodiments of the invention, ABCB5(+) stem cells are synthetic stem cells. ABCB5+ stem cells isolated from human tissue can be passaged in culture to produce populations of cells that are structurally and functionally distinct from the original primary cells isolated from the tissue. These cells are referred to herein as synthetic or manufactured ABCB5+ stem cells. These cells are in vitro manufactured such that nearly all cells are in vitro progeny of physiologically occurring skin-derived ABCB5-positive mesenchymal stem cells that never existed in the context of the human body. Rather, they are newly created. The compositions of the invention are populations of cells. The term “population of cells” as used herein refers to a composition comprising at least two, e.g., two or more, e.g., more than one, synthetic ABCB5+ stem cells, and does not denote any level of purity or the presence or absence of other cell types, unless otherwise specified. In an exemplary embodiment, the population is substantially free of other cell types. In some embodiments greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells.

The synthetic cells may also have distinct gene expression profiles relative to primary stem cells isolated from human tissue. The populations of synthetic cells (also referred to as ABCB5+ cells isolated from high passages) are different from the primary cells (those derived from low passage cultures that contain the native ABCB5+ cells found in the living organism). For example, certain stem cell markers are increased in high passage cells, e.g. SOX2, NANOG and SOX3, while certain mesenchymal stromal differentiation markers are decreased, e.g. MCAM, CRIG1 and ATXN1. The expression of selected stemness markers such as SSEA-4, DPP4 (CD26), PRDM1 (BLIMP1) and POU5F1 (OCT-4) in ABCB5+ cells in human skin at protein level was confirmed by immunostaining. While the expression of lower fibroblast lineage marker α-smooth muscle actin (α-SMA) was absent in ABCB5+ cells of human skin. These data support the finding that these late passage synthetic cells maintain pluripotent properties of ABCB5+ cells, and even have enhanced properties relative to the original cells.

In some preferred embodiments, 100% of the cells are synthetic, with 0% of the cells originating from the human tissue.

The ABCB5+ stem cells used herein are preferably isolated. An “isolated ABCB5+ stem cell” as used herein refers to a preparation of cells that are placed into conditions other than their natural environment. The term “isolated” does not preclude the later use of these cells thereafter in combinations or mixtures with other cells or in an in vivo environment.

The ABCB5+ stem cells may be prepared as substantially pure preparations. The term “substantially pure” means that a preparation is substantially free of cells other than ABCB5 positive stem cells. For example, the ABCB5 cells should constitute at least 70 percent of the total cells present with greater percentages, e.g., at least 85, 90, 95 or 99 percent, being preferred. The cells may be packaged in a finished pharmaceutical container such as an injection vial, ampoule, or infusion bag along with any other components that may be desired, e.g., agents for preserving cells, or reducing bacterial growth. The composition should be in unit dosage form.

The ABCB5+ stem cells may be autologous to the host (obtained from the same host) or non-autologous such as cells that are allogeneic or syngeneic to the host. Non-autologous cells are derived from someone other than the patient. Alternatively the ABCB5+ stem cells can be obtained from a source that is xenogeneic to the host.

Allogeneic refers to cells that are genetically different although belonging to or obtained from the same species as the host or donor. Thus, an allogeneic human mesenchymal stem cell is a mesenchymal stem cell obtained from a human other than the intended recipient of the ABCB5+ stem cells. Syngeneic refers to cells that are genetically identical or closely related and immunologically compatible to the host or donor, i.e., from individuals or tissues that have identical genotypes. Xenogeneic refers to cells derived or obtained from an organism of a different species than the host or donor.

In all cases an effective dose of cells should be given to a patient. The number of cells administered should generally be in the range of 1×10⁷-1×10¹⁰ and, in most cases should be between 1×10⁸ and 5×10⁹. Actual dosages and dosing schedules will be determined on a case by case basis by the attending physician using methods that are standard in the art of clinical medicine and taking into account factors such as the patient's age, weight, and physical condition. The cells will usually be administered by intravenous injection or infusion although methods of implanting cells may be used as well.

The ABCB5+ stem cells are useful in the treatment of liver disease. Liver disease includes disease such as hepatitis which result in damage to liver tissue. More generally, the ABCB5+ stem cells of the present invention can be used for the treatment of hepatic diseases, disorders or conditions including but not limited to: alcoholic liver disease, hepatitis (A, B, C, D, etc.), focal liver lesions, primary hepatocellular carcinoma, large cystic lesions of the liver, focal nodular hyperplasia granulomatous liver disease, hepatic granulomas, hemochromatosis such as hereditary hemochromatosis, iron overload syndromes, acute fatty liver, hyperemesis gravidarum, intercurrent liver disease during pregnancy, intrahepatic cholestasis, liver failure, fulminant hepatic failure, jaundice or asymptomatic hyperbilirubinemia, injury to hepatocytes, Crigler-Najjar syndrome, Wilson's disease, alpha-1-antitrypsin deficiency, Gilbert's syndrome, hyperbilirubinemia, nonalcoholic steatohepatitis, porphyrias, noncirrhotic portal hypertension, noncirrhotic portal hypertension, portal fibrosis, schistosomiasis, primary biliary cirrhosis, Budd-Chiari syndrom, hepatic veno-occlusive disease following bone marrow transplantation, etc.

Another use for the ABCB5+ stem cells of the invention is in liver tissue regeneration. In this aspect of the invention, the ABCB5 positive cells are used to generate liver tissue by induction of differentiation. Isolated and purified synthetic ABCB5+ stem cells can be grown in an undifferentiated state through mitotic expansion in a hepatocytic specific medium. These cells can then be activated to differentiate into tissue in vivo, as shown in the examples.

The methods and devices of the invention utilize isolated dermal mesenchymal and limbal progenitor cells as well as synthetic cells which, under certain conditions, can be induced to differentiate into and produce liver tissue.

The ABCB5+ stem cells may be modified to express proteins which are also useful in the therapeutic indications, as described in more detail below. For example, the cells may include a nucleic acid that produces at least one bioactive factor which further induces or accelerates the differentiation of the synthetic ABCB5+ stem cells into a differentiated lineage.

As used herein, a subject is a human, non-human primate, cow, horse, pig, sheep, goat, dog, cat or rodent. Human dermal synthetic ABCB5+ stem cells and human subjects are particularly important embodiments.

In a still further aspect of the invention described herein, ABCB5+ stem cells may be genetically engineered (or transduced or transfected) with a gene of interest. The transduced cells can be administered to a patient in need thereof, for example to treat genetic disorders or diseases.

The ABCB5+ stem cells, and progeny thereof, can be genetically altered. Genetic alteration of an ABCB5+ stem cell includes all transient and stable changes of the cellular genetic material which are created by the addition of exogenous genetic material. Exogenous genetic material includes nucleic acids or oligonucleotides, either natural or synthetic, that are introduced into the ABCB5+ stem cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include transfection of nucleic acid CaPO4 precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization in cycling, proteins that target intracellular localization and enhance intracellular half-life, and the like. Polymeric delivery systems also have been used successfully to deliver nucleic acids into cells, as is known by those skilled in the art. Such systems even permit oral delivery of nucleic acids.

One method of introducing exogenous genetic material into the ABCB5+ stem cells is by transducing the cells using replication- deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art.

A major advantage of using retroviruses is that the viruses insert efficiently a single copy of the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of ABCB5+ stem cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target mesenchymal stem cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into dermal synthetic ABCB5+ stem cells. The selection of an appropriate vector to deliver a therapeutic agent for a particular condition amenable to gene replacement therapy and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works contiguous with the coding sequence (in cis) to change the basal transcription level dictated by the promoter. Preferably, the exogenous genetic material is introduced into the dermal mesenchymal stem cell genome immediately downstream from the promoter so that the promoter and coding sequence are operatively linked so as to permit transcription of the coding sequence. A preferred expression vector includes an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eukaryotic cells.

These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified dermal mesenchymal stem cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the subject.

In addition to at least one promoter and at least one heterologous nucleic acid encoding the therapeutic agent, the expression vector preferably includes a selection gene, for example, a neomycin resistance gene, for facilitating selection of ABCB5+ stem cells that have been transfected or transduced with the expression vector. Alternatively, the ABCB5+ stem cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation.

The selection and optimization of a particular expression vector for expressing a specific gene product in an isolated stem cell is accomplished by obtaining the gene, preferably with one or more appropriate control regions (e.g., promoter, insertion sequence); preparing a vector construct comprising the vector into which is inserted the gene; transfecting or transducing cultured dermal synthetic ABCB5+ stem cells in vitro with the vector construct; and determining whether the gene product is present in the cultured cells.

Thus, the present invention makes it possible to genetically engineer ABCB5+ stem cells in such a manner that they produce polypeptides, hormones and proteins not normally produced in human stem cells in biologically significant amounts or produced in small amounts but in situations in which overproduction would lead to a therapeutic benefit.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1

### Human Skin-Derived ABCB5⁺ Stem Cell Injection Improves Liver Disease Parameters in Mdr2KO Mice

Although liver transplantation is a potential effective cure for patients with end-stage liver diseases, this strategy has several drawbacks including high cost, long waiting list, and limited availability of liver organs. Therefore, stem cell-based therapy is presented as an alternative option, which showed promising results in animal models of acute and chronic liver injuries. ABCB5⁺ cells isolated from skin dermis represent an easy to access and expandable source of homogenous stem cell populations. They showed already promising results in the treatment of corneal and skin injury. To date, the effect of these cells on liver injury is still unknown. It is shown that cell injections into fibrotic Mdr2KO mice as well as into mice upon partial liver resection, have no signs of toxicity with regard to cell transformation, cellular damage, fibrosis or inflammation. The effects of ABCB5⁺ cells on established biliary liver fibrosis in the Mdr2KO mice were investigated next. ABCB5⁺ cells influenced the shape of the liver inflammatory response to some extent and significantly reduced the amount of collagen deposition. Furthermore, a tendency of reduced apoptosis and enhanced death compensatory proliferation resulted from ABCB5⁺ cell transformation. The stem cells secreted several trophic factors that activated TGF-β family signalling in cultured LX-2 hepatic stellate cells (HSCs), therewith shaping cell fate to an αSMA low, Vimentin high phenotype. Taken together, ABCB5⁺ cells can represent a safe and feasible strategy to support liver regeneration and reduce liver fibrosis in chronic liver diseases.

A new subpopulation of skin stem cells characterized by expressing ATP-binding cassette sub-family B, member 5 (ABCB5) P-glycoprotein, which plays a role in their cell fusion has been identified. ABCB5⁺ dermal cells were proven to have immunomodulatory effects and prolonged allograft survival in a murine model of major histocompatibility complex (MHC)-mismatched cardiac allotransplantation by suppressing T-cell proliferation through programmed deathl (PD-1) [17]. Additionally, ABCB5⁺ cells mediated resistance to chemotherapy in Merkel cell carcinoma [18], and their transplantation protected against neutrophil-skin injury and improved survival in immunodeficient Col7α1KO mice, a model for recessive dystrophic epidermolysis bullosa (RDEB) [19, 20]. Besides the skin, ABCB5⁺ cells were detected also in other tissues such as placenta [21] and as part of the limbal stems cells (LSC) [22, 23]. In

LSC-deficient mice, transplanted ABCB5⁺ LSCs were able to fully restore the cornea [22].

In a recent study, ABCB5⁺ stem cells did not acquire sufficient characteristics of hepatocytes upon transdifferentiation experiments in vitro. The therapeutic effects of ABCB5⁺ cells on liver fibrosis were previously unknown. The impact of human skin dermis-derived ABCB5⁺ cells in the multidrug resistance gene2 (Mdr2) knockout mouse model is demonstrated herein.

Mdr2KO mice represent a common liver disease model of inflammatory biliary fibrosis that resembles primary sclerosing cholangitis (PSC) [25, 26]. Mechanistically, knockout of the Mdr2 leads to complete absence of phospholipids in bile. Phospholipid deficiency results in biliary damage, which spontaneously progresses to severe biliary fibrosis and hepatocellular carcinoma (HCC) [27]. Here, the effects of intravenously administrated ABCB5⁺ cells on inflammation, fibrosis, and apoptosis in 16 weeks old Mdr2KO mice presenting with F1 stage fibrosis, with and without immunosuppressive drug, are evaluated.

### Materials and Methods

### Reagents

Chemical reagents were purchased from Sigma Aldrich (Munich, DE), unless otherwise stated. Primer sequences and antibodies are listed in Tables 1 and 2, respectively.

### Preparation of ABCB5⁺ Mesenchymal Stromal Cells

ABCB5⁺ cells are prepared at TICEBA GmbH labs (Heidelberg, DE). Briefly, healthy skin specimens are collected from human donors following the Declaration of Helsinki principles. ABCB5⁺ cells are freshly isolated from expanded cell cultures using magnetic activated cell sorting (MACS) technology (GMP manufacturing process with customized anti-ABCB5 antibody coupled to customized magnetic beads) as described by Frank et al. [16]. Isolated cells are counted and suspended in 49.5% Ringer's lactate solution plus 2.5% human serum albumin (HSA, Octapharma GmbH Langenfeld, DE), and 0.4% glucose. In the present project, cells are injected into the tail vein of mice as single or double dosage of 5×10⁵ ABCB5⁺ cells each.

### Cell Culture and Treatment

Human stellate cells (LX-2) are seeded at 3×10⁵/well in six-well plates with Dulbecco's Modified Eagle Medium (DMEM, Biozym, Hessisch Oldendorf, DE), supplemented with 2% fetal bovine serum (FBS, Invitrogen, Karlsruhe, DE), 1% penicillin/streptomycin, and 1% glutamine. After six hours, medium is replaced by starvation medium, i.e. growth medium without FBS. At the next day, LX-2 cells are treated for the indicated time frame with 10 μmol transforming growth factor (TGF)-β1 (Peprotech, Rocky Hill, USA), 10 μmol LY2157299 (LY, Selleckchem, Houston, USA) or supernatant from ABCB5⁺ cells cultured under different conditions, as follows.

Before supernatant collection, 2×10⁵ ABCB5⁺ cells were seeded and stimulated either with 50 IU/ml Interferon-gamma (INF-γ, Boehringer Ingelheim, Ingelheim, DE) plus 20 ng/ml lipopolysaccharide from Escherichia coli (LPS), or were co-cultured with 1×10⁵ cells of the THP-1 macrophage cell line, pre-activated by phorbol 12-myristate 13-acetate (PMA) and stimulated with INF-γ and LPS. Upon treatment, protein lysate of LX-2 cells was collected after 1 and 24 h, whereas RNA samples were obtained after 2 and 24 h.

### Animal Model and Treatment

Mdr2KO mice with Balb/c background were kindly provided by Prof. Frank Lammert (Saarland University, Homburg) [25] and kept under specific-pathogen-free conditions in a fixed 12 h light/dark cycle. Mice were fed normal chow and water ad libitum. All animal experiments were performed according to the international guidelines with prior approval from regulatory authorities (Regierungspräsidium Karlsruhe). Genotyping of the mice was done as previously described [28, 29] using the following primer pair: Forward: GCTGAGATGGATCTTGAG Reverse: GTCGAGTAGCCAGATGATGG. Mice of both genders were used at the age of 16 weeks and randomized into control and ABCB5⁺ cells treated groups (n=4-8). To test the efficacy of ABCB5⁺ cells against Mdr2KO-induced liver fibrosis, three treatment strategies were utilized as follows:

Experiment 1: Animals were immunosuppressed with tacrolimus using a releasing pump, which was implanted on the back of the mice (isMdr2KO). Two days after pump implantation, mice received a single dosage of 5×10⁵ ABCB5⁺ cells or 100 μl NaCl. Mice were sacrificed for analyses of the effect of ABCB5⁺ stem cells two days, two weeks and four weeks after cell administration.

Experiment 2: Animals were immunosuppressed with tacrolimus and injected with two dosages of 5×10⁵ ABCB5+ cells at days 3 and 10 after pump implantation. Mice were sacrificed one week after the last injection of ABCB5⁺ cells.

Experiment 3: ABCB5⁺ cells were injected without prior immunosuppression of the Mdr2KO mice (nisMdr2KO). Here, blood and liver samples were collected 6 days after cell administration.

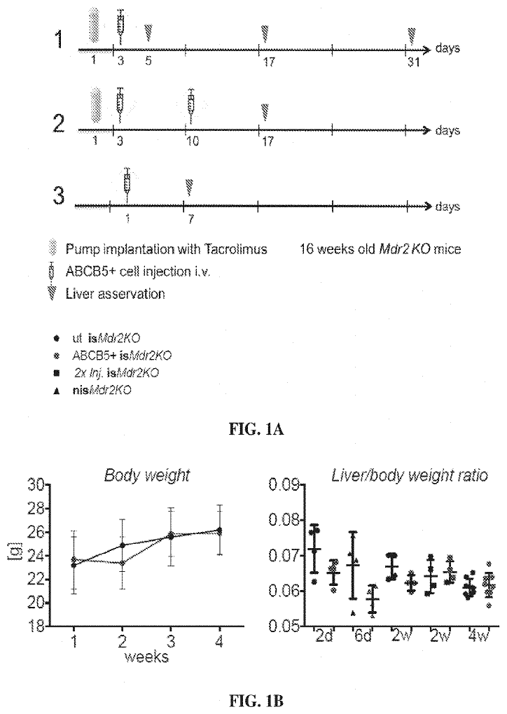

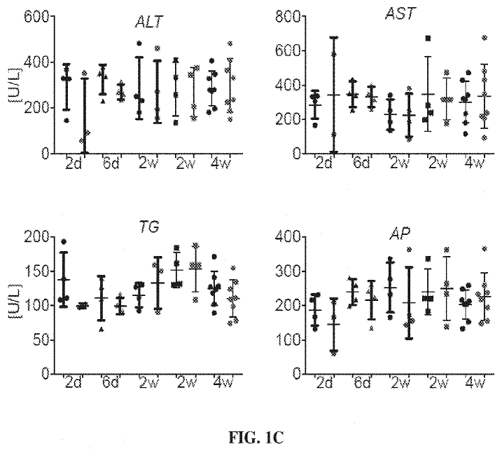

Saline-treated mice were always included as controls. The experimental setup is summarized in FIG. 1A.

Osmotic Pump Preparation and Implantation

A Mini-osmotic pump, model 1002 (Alzet®, Cupertino, USA) was used for constant release of drug for 2, 7 and 14 days, whereas the model 2004 was used for 4 weeks delivery. The pumps were filled according to manufacturer's instructions. Briefly, 5 mg Tacrolimus (Astellas, Munich, DE) diluted in 0.9% NaCl was filled slowly into the Alzet® pump under sterile conditions, at room temperature (RT) one day before implantation. A saline-filled osmotic pump was implanted in the control groups. In both models used, the release rate was adjusted to 1 mg/kg/day. After filling, pumps were primed in sterile saline overnight at RT prior to implantation to ensure immediate pumping. For implantation surgery, mice were anesthetized with 3% isoflurane and the site of pump implantation was shaved and disinfected with 70% ethanol. A half centimeter-mid-scapular incision was made and widened with a hemostat to create a suitable pocket for the pump. Filled pumps were inserted into the pocket with the delivery portal away from the incision site. Finally, the wound was closed with tissue adhesive (Surgibond®) and a single suture (Vicryl 6-0, Ethicon, New Jersey, USA). To decrease postoperative pain, mice were injected intraperitoneal (I.P) with 5 mg/kg carprofen (Pfizer, Karlsruhe, DE).

Analysis of Liver Function Parameters in Plasma

Under 3% isoflurane anaesthesia, retrobulbar blood was collected and centrifuged at 14,000 rpm for 3 min at 4° C. Plasma was transferred to new reaction tubes and stored at −20° C. Liver function parameters, i.e. alanine aminotransferase (ALT), aspartate aminotransferase (AST), alkaline phosphatase (AP), glucose, cholesterol, triglycerides (TG), protein bilirubin, and glutamate dehydrogenase (GLDH) were measured using the Olympus AU 400 automatic analyser.

Quantitative Real-Time RT-PCR (qPCR)

Total RNA was isolated from tissue of the right middle liver lobes using an InviTrap® Spin Universal RNA Mini Kit (Stratec, Birkenfeld, DE) according to manufacturer's instruction. RNA concentration was measured with the Infinite M200 Microplate reader (Tecan, Männedorf, CH). From 1 μg total RNA, cDNA was transcribed with RevertAid H Minus Reverse Transcriptase (Thermo Fischer Scientific, Massachusetts, USA) and used for qPCR with Power SYBR Green (Life Technologies, California, USA) in a StepOne Plus PCR System (Applied Biosystems, Foster City, USA). Corresponding primer pairs are listed in Table 1. Ppia was used as internal reference gene. Target genes were determined with the ΔΔCt method [30] and a melt curve was created to ensure primer specificity. Transcript numbers in each sample/experiment were quantified in triplicates.

TABLE 1
Sequences of primer pairs used for real time PCR

| | | SEQ | | SEQ |
|---|---|---|---|---|
| Genes | Forward | ID NO | Reverse | ID NO |
| αSma | TTCGCTGTCTACCTTCCAGC | 1 | GAGGCGCTGATCCACAAAAC | 19 |
| αVegf | AAGCGCAAGAAATCCCGGTT | 2 | GCTTGTCACATCTGCAAGTACG | 20 |
| B2M | TCTACTGGGATCGAGACATGT | 3 | ATTGCTATTTCTTTCTGCGTGCAT | 21 |
| | GA | | | |
| Casp3 | AGCTTGGAACGGTACGCTAA | 4 | GAGTCCACTGACTTGCTCCC | 22 |
| Casp9 | ACTGCCAAGAAAATGGTCACG | 5 | CAATGGACACGGAGCATCCA | 23 |
| Col1α1 | ACGTGGAAACCCGAGGTATG | 6 | TTGGGTCCCTCGACTCCTAC | 24 |
| Ctgf | AGATTGGAGTGTGCACTGCCA | 7 | TCCAGGCAAGTGCATTGGTATTTG | 25 |
| | AAG | | | |
| Il-1β | CCCAACTGGTACATCAGCACC | 8 | GACACGGATTCCATGGTGAAGTC | 26 |
| | TC | | | |
| Il-1β | TGCCACCTTTTGACAGTGATG* | 9 | TGATGTGCTGCTGCGAGATT* | 27 |
| Il-6 | TAGTCCTTCCTACCCCAATTTC | 10 | TTGGTCCTTAGCCACTCCTTC | 28 |
| | C | | | |
| Pparγ | TCCAGCATTTCTGCTCCACA | 11 | ACAGACTCGGCACTCAATGG | 29 |
| Ppia | GAGCTGTTTGCAGACAAAGTC | 12 | CCCTGGCACATGAATCCTGG | 30 |
| Il1rα | AGTACTGCCGAGGCCTGTAAT | 13 | TTGTTCCTCAGGCCCCAAT | 31 |
| | AA | | | |
| Tata | TCTGGAATTGTACCGCAGCTT | 14 | ATGACTGCAGCAAATCGCTTG | 32 |
| Tgfβ1 | AGGGCTACCATGCCAACTTC | 15 | CCACGTAGTAGACGATGGC | 33 |
| Timp1 | CGAGACCACCTTATACCAGCG | 16 | ATGACTGGGGTGTAGGCGTA | 34 |
| Tnfα | CCCTCACACTCACAAACCAC | 17 | ATAGCAAATCGGCTGACGGT | 35 |
| Tnfα | GTAGCCCACGTCGTAGCAAA* | 18 | ACAAGGTACAACCCATCGGC* | 36 |

* These primers were used exclusively in partial liver resection analysis

### Determination of Hepatic Hydroxyproline Content

Total hydroxyproline (HYP) as a measure of liver collagen content was estimated spectrophotometrically according to previously described protocols [31, 32].

### Histology, Histochemistry and Immunohistochemistry (IHC)

Liver tissues were fixed in 4% formaldehyde and embedded in paraffin. Three micrometer-liver tissue sections were cut for histological and immunohistochemical staining. Collagen content of the liver was determined by sirius red staining, as previously described [33, 34]. Briefly, liver sections were deparaffinized in xylene, rehydrated in serial dilutions of ethanol, and stained with sirius red (0.1% W/V sirius red F3B (C.I. 35782), dissolved in saturated aqueous solution of picric acid) for 1 h at RT. Next, slides were washed twice in absolute ethanol, mounted with malinol, covered, and left to dry. Immunohistochemistry (IHC) was performed as described [35]. After deparaffinization, antigen retrieval was performed in either EDTA buffer (1 mmol/L, pH 8.0) for 10 min, citrate buffer (10 mmol/L, pH 6.0) for 14 min, or in presence of proteinase K (Dako, Hamburg, DE) for 5 min, depending on the antigen (Table 2). Slides were incubated with endogenous peroxidase blocker (Dako, Hamburg, DE) for 20 min at RT. For F4/80, CD45, and Ki-67 antibodies, slides were additionally incubated for 15 min in avidin blocking solution, followed by biotin blocking (Abcam, Cambridge, UK) for 15 min. After washing with phosphate-buffered saline (PBS), sections were incubated with primary antibodies at 4° C. overnight (Table 2). The next day, slides were washed with PBS and incubated with secondary antibodies for 1 h at RT. Brown color was developed with diaminobenzidine (DAB), and nuclei were counterstained with hematoxylin (Merck, Darmstadt, DE). For quantification, 15 pictures of 10× non-overlapping fields (5 of the central area, 5 of the portal area, and 5 of the mid-zonal area) were taken by Leica microscope (Leica Microsystem, Wetzlar, Germany) and analyzed by ImageJ software 1.48v (NIH, Maryland, USA).

### One Third Partial Liver Resection

Under deep anesthesia, the surgical handling to remove the left lateral lobe was essentially the same as described in (The generation of hepatocytes from mesenchymal stem cells and engraftment into murine liver. Stock P, Brückner S, Ebensing S, Hempel M, Dollinger M M, Christ B. Nat. Protoc. 2010;5:617-627.). Briefly, the abdomen was opened along the linea alba. The exposed lobe was ligated, and removed. After closure of the abdominal cavity, another 1 cm incision along the lower costal arch was performed to expose the spleen. After ligation beneath the apical spleen pole, 150 μl of 7.5×10⁵ ABCB5⁺ stem cells or 150 μl HRG (49.5 vol.-% of 5% human serum albumin; 49.5 vol.-% of Ringer-lactate; 1 vol.-% of 4% glucose) as carrier control were injected into the ligated spleen pole. The incision was closed, 500 μl of 0.9% NaCl solution were delivered subcutaneously for volume substitution, and 100 mg per kg body weight of piperacillin (Fresenius Kabi GmbH, Bad Homburg, Germany) was administered for antibiosis. Anesthesia was stopped, and animals kept for recovery.

TABLE 2
Primary* and secondary antibodies used in the study

| Antibody | | Product | | Appli- | Dilu- |
|---|---|---|---|---|---|
| name | Host | number | Company | cation | tion |
| F4/80 | Rat | MCA497R | Biorad | IHC | 1:2000 |
| CD45 | Rat | 550539 | BD | IHC | 1:100 |
| | | | Bioscience | | |
| αSMA | Mouse | m0851 | Dako | IHC | 1:1000 |
| CD163 | Rabbit | ab182422 | Abcam | IHC | 1:500 |
| SM22α | Rabbit | ab14106 | Abcam | IHC | 1:100 |
| KI-67 | Rabbit | 12202s | Cell | IHC | 1:250 |
| | | | signaling | | |
| Biotinylated | Goat | BA-9400 | Vector | IHC | 1:2000 |
| Anti-Rat | | | Lab | | (F4/80) |
| IgG | | | | | |
| | | | | | 1:500 |
| | | | | | (CD45) |
| Biotinylated | Goat | ab6720 | Abcam | IHC | 1:500 |
| Anti-Rabbit | | | | | |
| IgG | | | | | |
| anti-mouse | Goat | p0447 | Dako | IHC | 1:200 |
| IgG HRP | | | | | |
| anti-rabbit | Goat | IMMRIR2932 | Immuno- | IHC | 1:200 |
| HRP | | | reagents | | |
| αSMA | Mouse | ab7817 | Abcam | WB | 1:1000 |
| Vimentin | Mouse | ab20346 | Abcam | WB | 1:1000 |
| pSMAD2 | Rabbit | 3101 | Cell | WB | 1:1000 |
| | | | Signaling | | |
| pSMAD3 | Rabbit | ab52903 | Abcam | WB | 1:1000 |
| GAPDH | Mouse | sc-47724 | Santa cruz | WB | 1:1000 |
| anti-rabbit | Mouse | SC2357 | Santa cruz | WB | 1:5000 |
| IgG HRP | | | | | |
| anti-mouse | Goat | SC2005 | Santa cruz | WB | 1:5000 |
| IgG HRP | | | | | |

*EDTA buffer was used for antigen retrieval with all IHC primary antibodies except for F4/80 where proteinase K was used and CD45 and KI-67 where citrate buffer was used.

Terminal Deoxynucleotidyl Transferase dUTP Nick End Labeling (Tunel) Assay

After deparaffinization, slides were incubated with Proteinase K for 20 min. After washing with PBS, tissues were covered with a 1:10 mixture of enzyme: label solution (In-situ cell death detection kit, Fluorescein, Roche, Mannheim, DE) and incubated at 37° C. in a humidified chamber in the dark. After 1 h, slides were washed with PBS and covered with fluorescent mounting medium (Dako, Hamburg, DE). Tissue sections with label solution only were included as negative control. For quantification, 5 pictures of 10× non-overlapping portal fields were taken by a Leica confocal microscope TCS SP8 (Leica Microsystem, Wetzlar, Germany) and quantified by ImageJ software 1.48v (NIH, Maryland, USA).

### Immunoblotting

Immunoblotting was done according to Mahmood and Yang 2012, with minor modifications [36]. Briefly, cells were lysed with Radioimmunoprecipitation assay (RIPA) buffer (20 mM Tris-HCl pH 7.5, 150 mM CaCl₂, 1% sodium deoxycholate, 1% Nonidet P-40, 0.1% SDS, 1 mM EDTA, 0.5 mM EGTA) in presence of Protease Inhibitor Cocktail and Phosphatase Inhibitor Cocktail II (Roche, Mannheim. DE). Cell lysate was centrifuged at 13,000 rpm for 10 min at 4° C., and supernatant was transferred to a new reaction tube. The protein concentration was determined with an Infinite M200 Microplate reader (Tecan, Männedorf, CH) using the DC protein assay (Bio-Rad, Munich, DE). Thirty micrograms protein were separated using sodium dodecyl sulfate-polyacrylamide gels (10%). After electrophoresis, proteins were transferred from gels to nitrocellulose membranes (Pierce, Rockford, Ill.) and blocked with 5% non-fat milk in Tris-buffered saline with Tween 20 (TBST buffer: 10 mM Tris, pH 8.0, 150 mM NaCl, and 0.5% Tween 20) for 2 h at RT. Afterwards, the membranes were incubated overnight at 4° C. with αSMA, Vimentin, pSMAD2, pSMAD3 or GAPDH primary antibodies (Table 2). The next day, membranes were washed with TBST buffer and incubated with secondary antibodies for 1 h at RT (Table 2). Finally, membranes were developed with a chemiluminescent substrate according to the manufacturer's instruction (Amersham, Freiburg, DE).

### Sirius Red Immunohistochemistry

Dewaxed paraffin slices were incubated in Sirius Red-solution (0.1 g Direct red 80 (Alfa Aesar, Karlsruhe, Germany); 100 ml picric acid) for 60 min. Then, slices were washed with 0.5% acetic acid (Carl Roth GmbH+Co. KG, Karlsruhe, Germany). After dehydration, slices were embedded in Entellan (Merck GmbH, Darmstadt, Germany). The percentage of Sirius Red positive collagen fibres was calculated in randomly defined microscopic areas using the ImageJ 1.48 v software (National Institute of Health, Bethesda, Md., USA).

### Semi-Quantitative RT-PCR

Total RNA was isolated from 40 mg of liver tissue in 750 μl QIAzol Lysis Reagent (Qiagen, Hilden, Germany) followed by chloroform extraction, isopropanol precipitation and cDNA synthesis (Maxima H Minus First cDNA syn Kit, Thermo Fischer, Dreieich, Germany). RT PCR was performed by using PCR-Mastermix 2× (Thermo Fischer, Dreieich, Germany) and the corresponding primer pairs as detailed in the Table. After electrophoretic separation in an agarose gel and staining with GelRed™, band intensity was quantified using the ImageJ 1.48 v software (National Institute of Health, Bethesda, Md., USA) and normalized to the expression of the housekeeping genes B2M and TATA.

### Statistics

Data were analyzed using GraphPad Prism version 6.0 for Windows (GraphPad Software Inc). Results were shown as mean±standard deviation (SD). Two-tailed unpaired Student t-test was used to compare between groups. Probability value (p) less than 0.05 was considered significant and represented graphically as *p<0.05; **p<0.01; ***p<0.001, unless otherwise indicated.

### Results

Hepatic Transplantation of ABCB5⁺ Cells does not Induce Liver Damage, Inflammation or Fibrosis

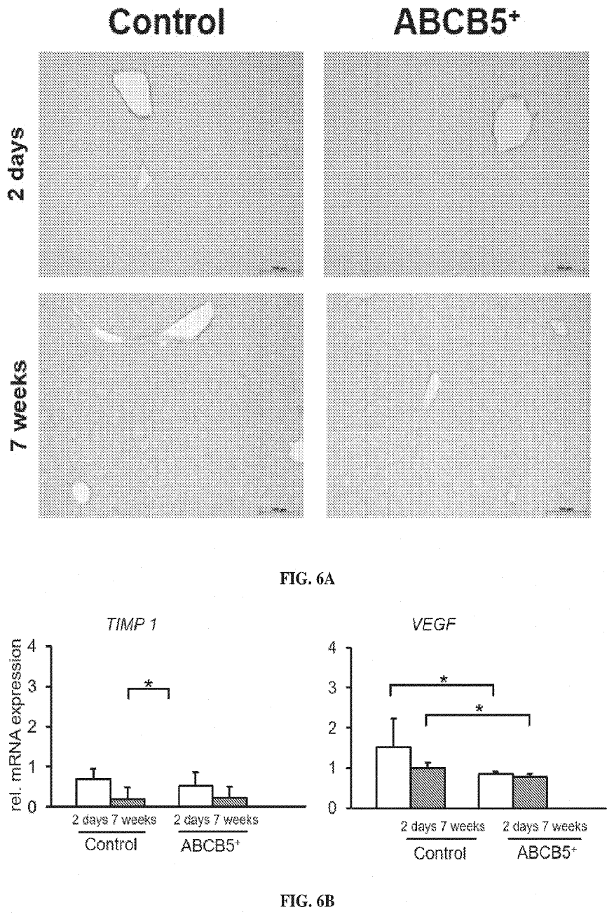

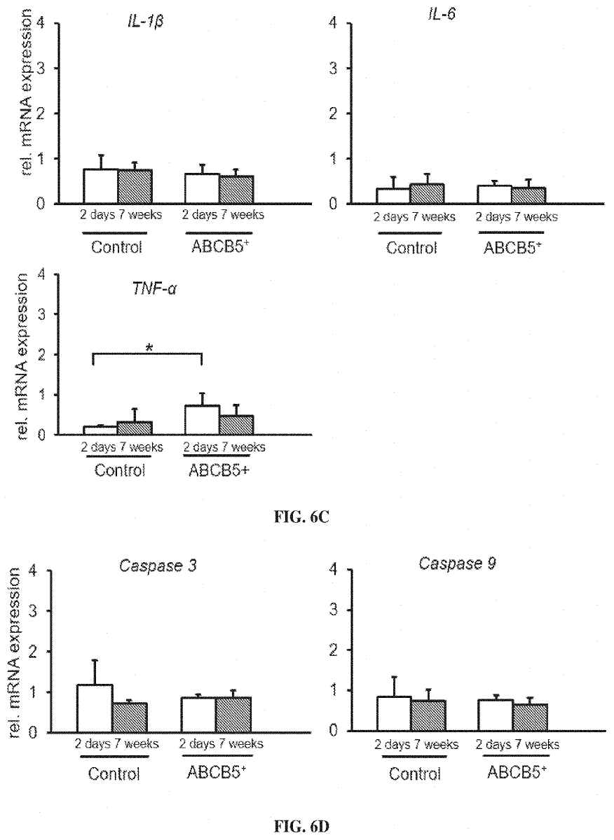

Staining of collagen fibers with Sirius red did not reveal significant differences between animals treated with the carrier control HRG or with ABCB5⁺ cells, neither at 2 days nor at 7 weeks after treatment (FIG. 6A). The decrease of the inhibitor of metalloproteinases TIMP1 (tissue inhibitor of metalloproteinases 1) is indicative of extracellular matrix remodeling associated with the pathogenesis of fibrosis. As assessed by semiquantitative RT-PCR, its expression was not significantly different in control and cell-treated animals. VEGF (vascular endothelial growth factor), which has been shown to be associated with angiogenesis during fibrosis (Role of hypoxia-inducible factors in the development of liver fibrosis. Roth K J, Copple B L. Cell Mol Gastroenterol Hepatol. 2015 Sep. 25;1(6):589-597), was significantly lower in animals receiving cell transplants than in control animals (FIG. 6B). The expression of the pro-inflammatory cytokines IL-1β and IL-6 was not different in control and cell-treated animals. At 2 days after cell transplantation, TNFα was higher in cell-treated animals as compared to controls, but decreased again to control levels 7 weeks after treatment (FIG. 6C). Caspases 3 and 9 were similar under all conditions tested, indicating no impact of the cell transplants on hepatocellular destruction (FIG. 6D). Also, the number of macrophages was similar at 2 days after treatment both in control and cell-treated animals as assessed by quantitative image analysis of macrophages after immunohistochemical visualization using the F4/80 antibody (not shown). Thus, transplantation of ABCB5⁺ did not cause any obvious adverse effects related to the pathogenesis of fibrosis or inflammatory processes, short- nor long-term.

ABCB5⁺ Cell Treatment does not Induce Toxicity in Mdr2KO Mice

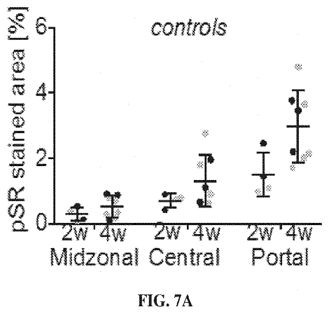

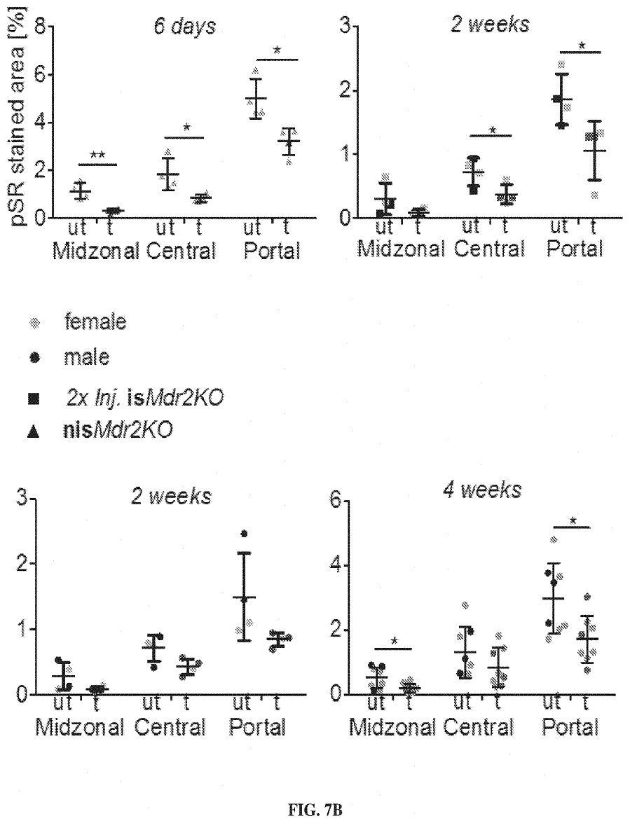

16 weeks old Mdr2KO mice on Balbc background and corresponding wt controls were used for cell transplantation experiments. At this age, the mice present with robust fibrosis and significant inflammation, representing an F1 disease stage [37, 38], and it was expected that disease modulating effects of the treatment would be visible. Furthermore, in contrast to studies with Mdr2KO mice on an XX background, increased fibrosis in untreated female mice was not diagnosed at ages 18 and 20 weeks, when the respective control samples were collected (FIG. 7A). Therefore, gender was not discriminated in all follow up analyses.

5×10⁵ ABCB5⁺ cells were injected into the tail vein of the mice in two alternate approaches. Either the mice were immunosuppressed by constant delivery of tacrolimus via a pump device to avoid possible rejection reactions (immunosuppressed, is), or this step was omitted in a second cohort (not immunosuppressed, nis), in line with the results of Schatton et al. [17], showing protective effects of ABCB5⁺ cells for cardiac allograft survival in the absence of immunosuppression. The livers were examined at different time points, i.e. at 2 d, 2 w and 4 w from isMdr2KO and 6 d from nisMdr2KO after ABCB5⁺ cell administration, as indicated in FIG. 1A (nisMdr2KO mice were examined only 6 d after ABCB5⁺ cell injection to limit the number of animal experiments, taking into account the 3R rule). The 2 d time point was used to search for presence of ABCB5³⁰ cells in the liver, whereas later time points, i.e. 6 d, 2 w, and 4 w were used to test a potential therapeutic benefit towards Mdr2KO-induced liver fibrosis. Also in this model, in both experiments, ABCB5⁺ cell therapy shows a good safety profile in terms of body weight, liver body weight ratio, and liver function parameters, including ALT, AST, AP, and TG (FIG. 1B, C), all values not being different from the saline-treated controls. As expected from the approach to inject the cells into the tail vein, evidence was not found for ABCB5⁺ cell settlement within the liver at any time point after injection.

### ABCB5⁺ Cell Treatment Reduces Collagen Deposition in Mdr2KO Mediated Biliary Fibrosis

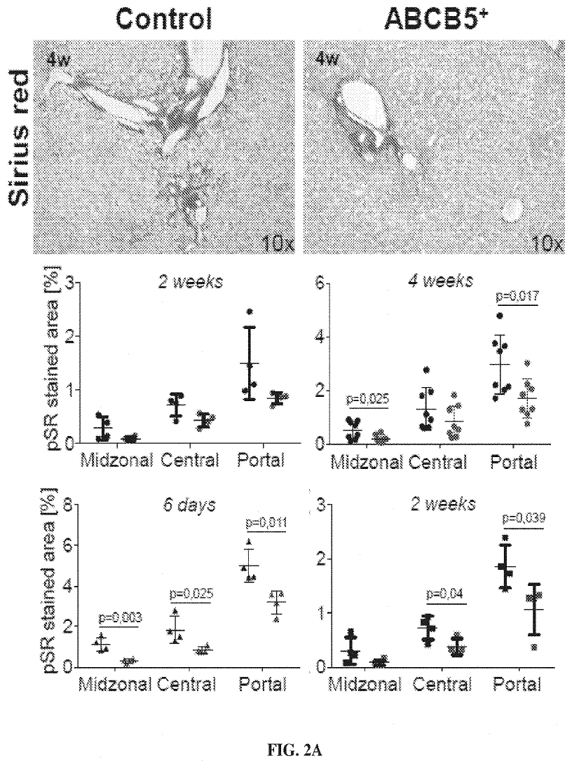

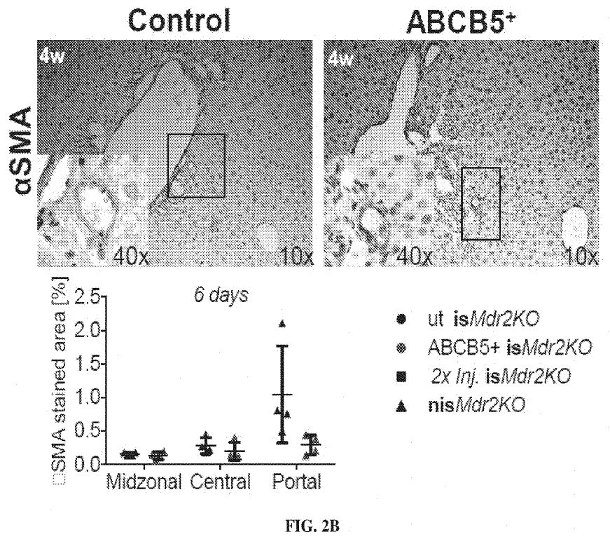

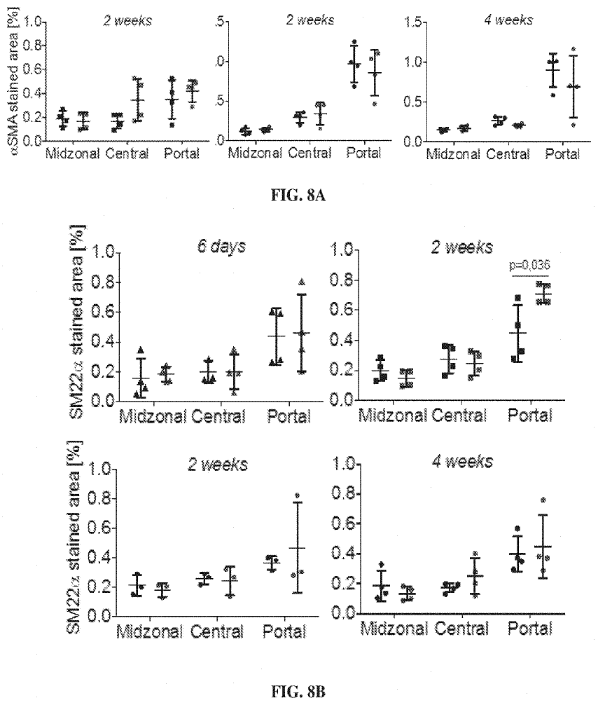

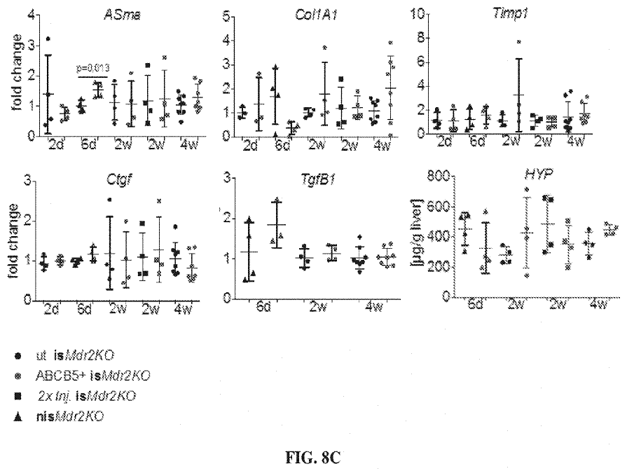

Next, liver fibrosis features were carefully investigated in the mice. Since in Mdr2KO mice, fibrogenesis is initiated and predominant in the portal areas and spreads from there to midzonal and pericentral areas, fibrosis was separately quantified in these regions and the quantification of Sirius red stain results was displayed as midzonal, central and portal. In isMdr2KO mice, ABCB5⁺ cell treatment significantly decreased deposition of collagen at the 4 weeks timepoint, particularly in the portal area (FIG. 2A). A tendency of reduced collagen deposition is already visible 2 weeks after cell injection, however significance was not reached at that time point. The data with nisMdr2KO were very similar to isMdr2KO, as well showing a pronounced effect on collagen deposition upon ABCB5⁺ cell treatment, which was most evident in the portal areas. A significant decrease of Sirius red staining was evident after 6 days, and after two weeks with two cell injections. In line with the above statement, the result is the same when male and female gender are discriminated (FIG. 7B) No significant change between the ABCB5⁺- cell treated and non treated groups was observed for alpha-smooth muscle actin (αSMA) and smooth muscle protein22-alpha (SM22α), markers of activated hepatic stellate cells (HSCs) and portal fibroblasts [39] (FIGS. 2B, 8A, 8B). The analysis showed threefold (αSMA) and twofold (SM22α) more positive staining in the damaged portal areas, as compared to central and mid-zonal areas in both, cell injected and control groups. A tendency of a reduced number of activated HSCs is only present in the 6 days nisMdr2KO treatment group (FIG. 2B), whereas for SM22α positive staining, a tendency of increased cell numbers at the two weeks double injection nisMdr2KO group is documented (FIG. 8B). Furthermore, no cell therapy dependent changes occured at mRNA levels of the typical fibrogenesis-related genes (FIG. 8C) connective tissue growth factor (Ctgf), tissue inhibitor of metalloproteinases (Timp)1, αSMA, transforming growth factor (Tgf)-β1, and collagen 1α1 (Col1α1). Similarly, hydroxyproline content was not altered after ABCB5⁺ cell administration, as measured in whole liver lysates.

### Effects of ABCB5⁺ Cells on Mdr2KO Induced Liver Inflammation

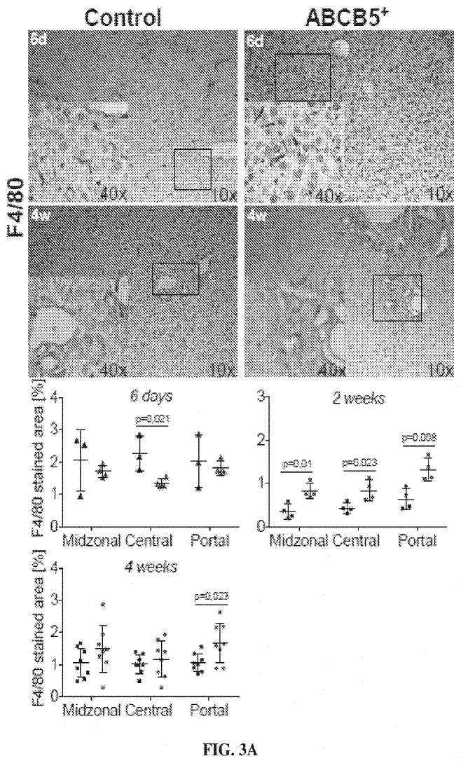

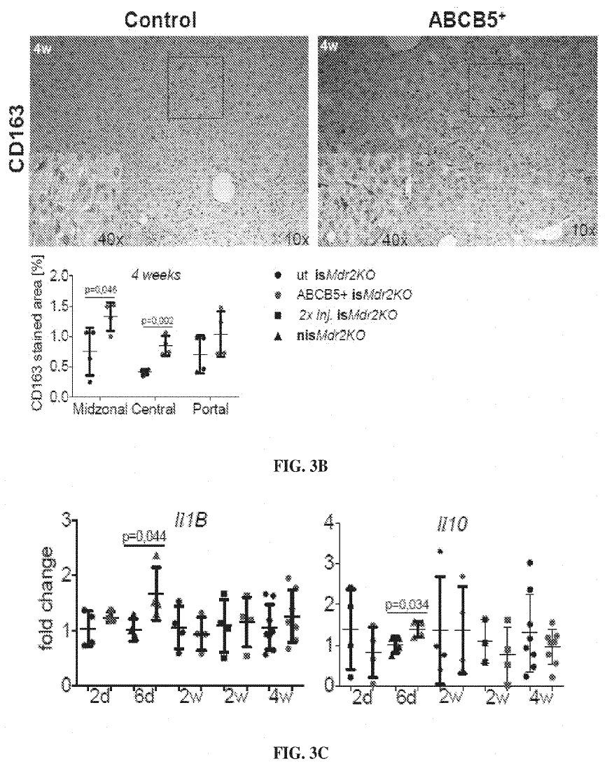

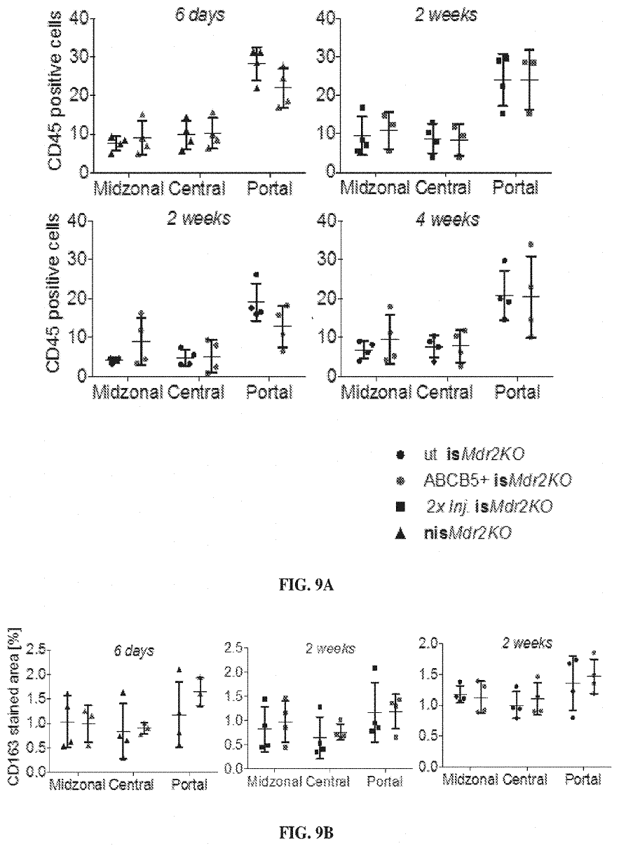

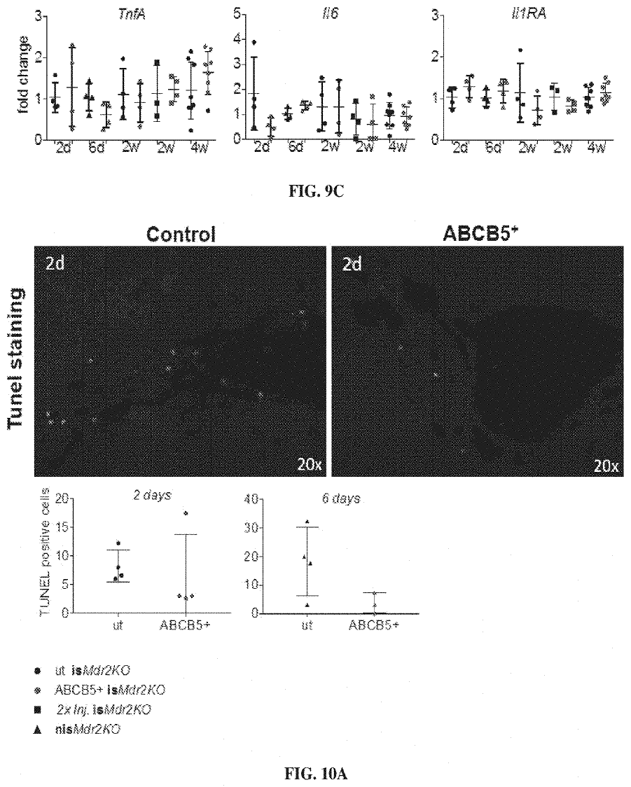

Because of the interdependence of liver inflammation and fibrosis [40], the inflammatory status of the liver with and without ABCB5⁺ cell treatment was compared. For this purpose, F4/80 was used as pan-macrophage marker, cluster of differentiation (CD163) as macrophage subtype 2 (M2) marker [41, 42], and CD45 as pan-leukocyte marker. Staining quantification results showed an increased number of F4/80⁺ (2 weeks and 4 weeks time points; FIG. 3A) and CD163⁺ cells (4 weeks time point; FIG. 3B; FIG. 9B) in ABCB5⁺ cells-treated immunosuppressed mice in comparison to controls. Noteworthy, after 4 weeks, this increase was significant mainly in the portal area in case of F4/80 staining, and in the mid-zonal and central area in case of CD163 staining. In contrast, F4/80⁺ cells were significantly decreased in the liver central zone of non-immunosuppressed mice, 6 days after ABCB5⁺ administration (FIG. 3A). Further, CD45 positive staining and mRNA levels of inflammatory genes, e.g. Il6, Tnfα, and Il1rα did not differ between ABCB5⁺ cells-treated mice and their corresponding controls in both immunosuppressed and non-immunosuppressed mice (FIGS. 9A, 9C). Interestingly, ABCB5⁺ cells increased proinflammatory Il1β and anti inflammatory Il10 mRNA expression levels significantly in non-immunosuppressed mouse livers (FIG. 3C).

### Effects of ABCB5⁺ Cells on Hepatocyte Apoptosis and Proliferation in MdrKO Mouse Model

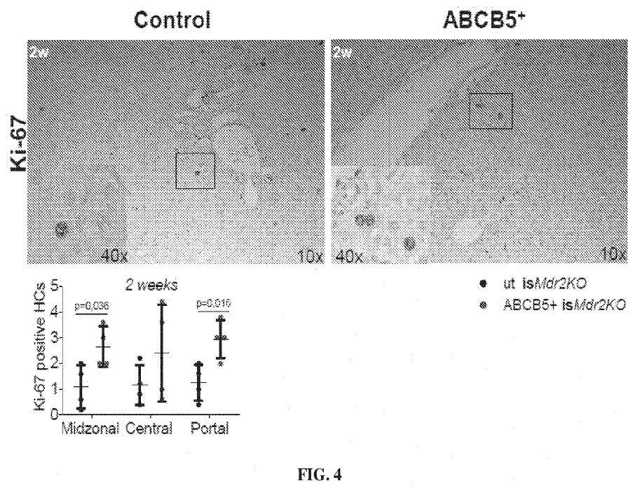

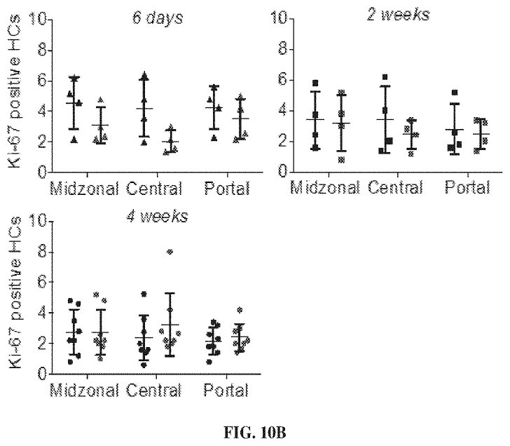

To test for hepatocyte death and regenerating hepatocyte proliferation, mouse liver tissue was stained for apoptosis and Ki-67. Only apoptosis was tested at early time points after cell injection and a tendency of reduction at 2 and 6 days was found, which however did not reach significance (FIG. 10A). 2 weeks upon ABCB5⁺ cell injection, non-immunosuppressed Mdr2KO mice display significantly increased numbers of Ki-67⁺ hepatocytes in portal and mid-zonal areas, as compared to the control group (FIG. 4), whereas all other samples did not show cell treatment-dependent differences (FIG. 10B). Further, there was no difference visible regarding Ki-67⁺ staining after treatment with ABCB5⁺ cells in non-parenchymal cells i.e., stellate cells, Kupffer cells, cholangiocytes, sinusoidal endothelial cells, and intrahepatic lymphocytes.

### Effects of the Secretome of ABCB5⁺ Cells on LX-2 Cells

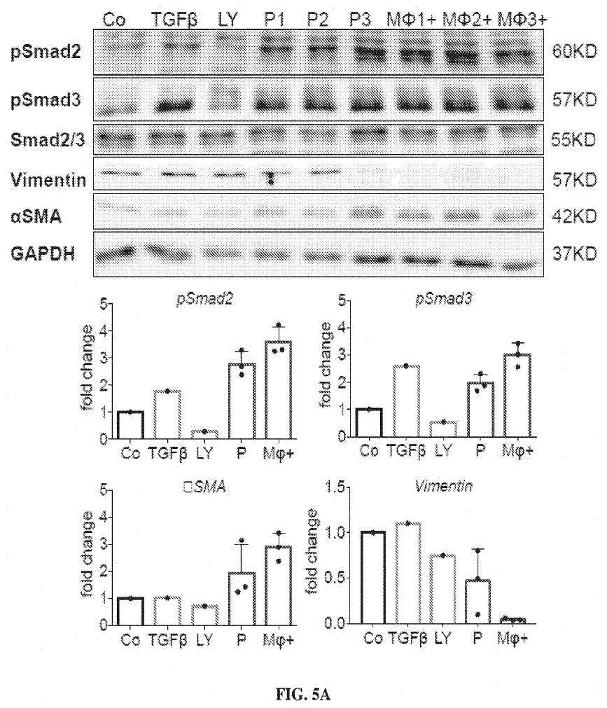

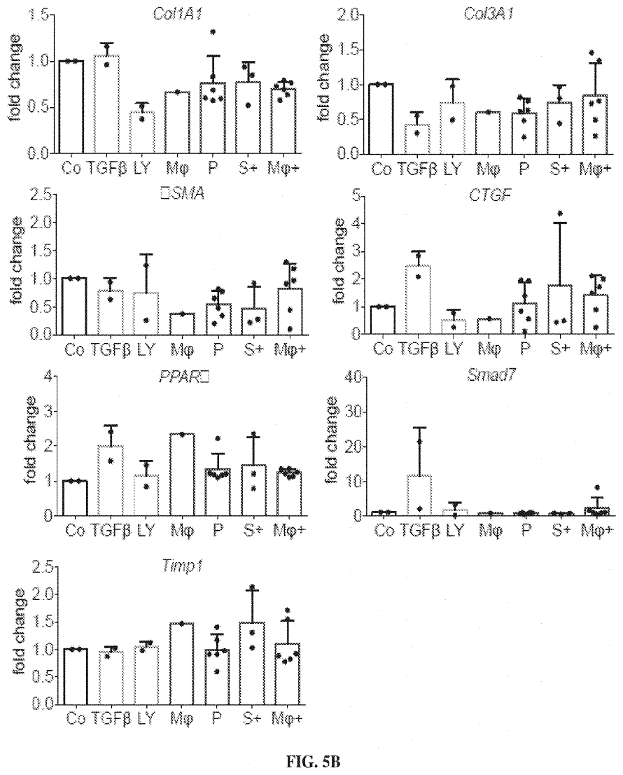

Because ABCB5⁺ stem cells were not detected in mice liver after injection (non-published results), it was speculated that ABCB5⁺ cells mediate their anti-fibrotic effects paracrinally through secretion of inflammatory mediators that affect the HSC transactivation status. To test this possible outcome, the human HSC cell line LX-2 was cultured with the supernatant of ABCB5⁺ cells that were previously stimulated or not with LPS, INF-γ, or THP-1 macrophages, as indicated in the figures. Interestingly, the supernatant of ABCB5⁺ cells induced αSMA production and decreased vimentin expression (FIGS. 5A, B). Moreover, the supernatant of ABCB5⁺ cells co-cultured with activated macrophages led to a further increase in αSMA protein expression and a further decrease in vimentin expression (FIGS. 5A, B). However, the mRNA levels of αSMA, Ctgf, Col1α1, Col3α1, Timp1, Pparγ, and Smad7 did not change in LX-2 cells cultured with/without the supernatant of ABCB5⁺ cells (FIG. 5C). The increased phosphorylation of SMAD2 and SMAD3 proteins suggests that this effect could be mediated through stimulating the TGF-β signaling pathway (FIGS. 5A, B). LX-2 cells treated with TGF-β1 recombinant protein and TGF-β1 signaling inhibitor, LY2157299, were included as positive and negative control of enhanced TGF-β signaling.

### REFERENCES

1. Song, A. T., et al., Liver transplantation: fifty years of experience. World J Gastroenterol, 2014. 20 (18): p. 5363-74.

2. Schuppan, D. and N. H. Afdhal, Liver cirrhosis. Lancet, 2008. 371(9615): p. 838-51.

3. European Association for the Study of the Liver. Electronic address, e.e.e., EASL Clinical Practice Guidelines: Liver transplantation. J Hepatol, 2016. 64(2): p. 433-485.

4. Library, E.S.R.; Available from: statistics.eurotransplant.org.

5. Margini, C., et al., Bone marrow derived stem cells for the treatment of end-stage liver disease. World J Gastroenterol, super einteilung!!!!!2014. 20(27): p. 9098-105.

6. Dhawan, A., et al., Human hepatocyte transplantation: current experience and future challenges. Nat Rev Gastroenterol Hepatol, 2010. 7(5): p. 288-98.

7. Meyburg, J., et al., One liver for four children: first clinical series of liver cell transplantation for severe neonatal urea cycle defects. Transplantation, 2009. 87(5): p. 636-41.

8. Baccarani, U., et al., Human hepatocyte transplantation for acute liver failure: state of the art and analysis of cell sources. Transplant Proc, 2005. 37(6): p. 2702-4.

9. Alwahsh, S. M., H. Rashidi, and D. C. Hay, Liver cell therapy: is this the end of the beginning? Cell Mol Life Sci, 2018. 75(8): p. 1307-1324.

10. Meier, R. P., et al., Transplantation of mesenchymal stem cells for the treatment of liver diseases, is there enough evidence? Stem Cell Res, 2013. 11(3): p. 1348-64.

11. Eom, Y. W., K. Y. Shim, and S. K. Baik, Mesenchymal stem cell therapy for liver fibrosis. Korean J Intern Med, 2015. 30(5): p. 580-9.

12. Suk, K. T., et al., Transplantation with autologous bone marrow-derived mesenchymal stem cells for alcoholic cirrhosis: Phase 2 trial. Hepatology, 2016. 64(6): p. 2185-2197.

13. Wang, Y. H., et al., Progress in mesenchymal stem cell-based therapy for acute liver failure. Stem Cell Res Ther, 2018. 9(1): p. 227.

14. Lee, C. W., et al., Historical Perspectives and Advances in Mesenchymal Stem Cell Research for the Treatment of Liver Diseases. Gastroenterology, 2018. 154(1): p. 46-56.

15. Wong, V. W., et al., Stem cell niches for skin regeneration. Int J Biomater, 2012. 2012: p. 926059.

16. Frank, N. Y., et al., Regulation of progenitor cell fusion by ABCB5 P-glycoprotein, a novel human ATP-binding cassette transporter. J Biol Chem, 2003. 278(47): p. 47156-65.

17. Schatton, T., et al., ABCB5 Identifies Immunoregulatory Dermal Cells. Cell Rep, 2015. 12(10): p. 1564-74.

18. Kleffel, S., et al., ABCB5-Targeted Chemoresistance Reversal Inhibits Merkel Cell Carcinoma Growth. J Invest Dermatol, 2016. 136(4): p. 838-46.

19. Webber, B. R., et al., Rapid generation of Col7a1−/−mouse model of recessive dystrophic epidermolysis bullosa and partial rescue via immunosuppressive dermal mesenchymal stem cells. Lab Invest, 2017. 97(10): p. 1218-1224.

20. Jiang, D., et al., Suppression of Neutrophil-Mediated Tissue Damage—A Novel Skill of Mesenchymal Stem Cells. Stem Cells, 2016. 34(9): p. 2393-406.

21. Volpicelli, E.R., et al., The multidrug-resistance transporter ABCB5 is expressed in human placenta. Int J Gynecol Pathol, 2014. 33(1): p. 45-51.

22. Ksander, B. R., et al., ABCB5 is a limbal stem cell gene required for corneal development and repair. Nature, 2014. 511(7509): p. 353-7.

23. Gonzalez, G., et al., Limbal stem cells: identity, developmental origin, and therapeutic potential. Wiley Interdiscip Rev Dev Biol, 2018. 7(2).

24. Tietze, L., et al., Assessment of the hepatocytic differentiation ability of human skin-derived ABCB5(+) stem cells. Exp Cell Res, 2018.

25. Lammert, F., et al., Spontaneous cholecysto- and hepatolithiasis in Mdr2−/−mice: a model for low phospholipid-associated cholelithiasis. Hepatology, 2004. 39(1): p. 117-28.

26. Popov, Y., et al., Mdr2 (Abcb4)−/−mice spontaneously develop severe biliary fibrosis via massive dysregulation of pro- and antifibrogenic genes. J Hepatol, 2005. 43(6): p. 1045-54.

27. Liedtke, C., et al., Experimental liver fibrosis research: update on animal models, legal issues and translational aspects. Fibrogenesis Tissue Repair, 2013. 6(1): p. 19.

28. Pikarsky, E., et al., NF-kappaB functions as a tumour promoter in inflammation-associated cancer. Nature, 2004. 431(7007): p. 461-6.

29. Mauad, T. H., et al., Mice with homozygous disruption of the mdr2 P-glycoprotein gene. A novel animal model for studies of nonsuppurative inflammatory cholangitis and hepatocarcinogenesis. Am J Pathol, 1994. 145(5): p. 1237-45.

30. Livak, K. J. and T. D. Schmittgen, Analysis of relative gene expression data using real-time quantitative PCR and the 2(-Delta Delta C(T)) Method. Methods, 2001. 25(4): p. 402-8.

31. Jamall, I. S., V. N. Finelli, and S. S. Que Hee, A simple method to determine nanogram levels of 4-hydroxyproline in biological tissues. Anal Biochem, 1981. 112(1): p. 70-5.

32. Boigk, G., et al., Silymarin retards collagen accumulation in early and advanced biliary fibrosis secondary to complete bile duct obliteration in rats. Hepatology, 1997. 26(3): p. 643-9.

33. Puchtler, H., F. S. Waldrop, and L. S. Valentine, Polarization microscopic studies of connective tissue stained with picro-sirius red FBA. Beitr Pathol, 1973. 150(2): p. 174-87.

34. Junqueira, L. C., G. Bignolas, and R. R. Brentani, Picrosirius staining plus polarization microscopy, a specific method for collagen detection in tissue sections. Histochem J, 1979. 11(4): p. 447-55.

35. Shen, Z., et al., Delta-Like Ligand 4 Modulates Liver Damage by Down-Regulating Chemokine Expression. Am J Pathol, 2016. 186(7): p. 1874-1889.

36. Liu, Z. Q., T. Mahmood, and P. C. Yang, Western blot: technique, theory and trouble shooting. N Am J Med Sci, 2014. 6(3): p. 160.

37. Hammad, S., et al., Galunisertib modifies the liver fibrotic composition in the Abcb4Ko mouse model. Arch Toxicol, 2018. 92(7): p. 2297-2309.

38. Katzenellenbogen, M., et al., Multiple adaptive mechanisms to chronic liver disease revealed at early stages of liver carcinogenesis in the Mdr2-knockout mice. Cancer Res, 2006. 66(8): p. 4001-10.

39. Vaahtomeri, K., et al., Lkb1 is required for TGFbeta-mediated myofibroblast differentiation. J Cell Sci, 2008. 121(Pt 21): p. 3531-40.

40. Pellicoro, A., et al., Liver fibrosis and repair: immune regulation of wound healing in a solid organ. Nat Rev Immunol, 2014. 14(3): p. 181-94.

41. Wijesundera, K. K., et al., M1- and M2-macrophage polarization in thioacetamide (TAA)-induced rat liver lesions; a possible analysis for hepato-pathology. Histol Histopathol, 2014. 29(4): p. 497-511.

42. Fabriek, B. O., C. D. Dijkstra, and T. K. van den Berg, The macrophage scavenger receptor CD163. Immunobiology, 2005. 210(2-4): p. 153-60.

All references cited herein are fully incorporated by reference. Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method for inducing liver tissue generation in a subject in need thereof, comprising injecting an isolated population of ABCB5+ stem cells into the subject in an effective amount to generate liver tissue.

2. A method for treating liver fibrosis in a subject in a subject in need thereof, comprising injecting an isolated population of ABCB5+ stem cells into the subject in an effective amount to treat liver fibrosis in the subject.

3. The method of any one of claims 1-2, wherein the ABCB5+ stem cells are cultured in a hepatocytic differentiation medium prior to injection into the subject.

4. The method of any one of claims 1-3, wherein the ABCB5+ stem cells are injected into the liver of the subject.

5. The method of any one of claims 1-3, wherein the ABCB5+ stem cells are injected systemically into the subject.

6. The method of any one of claims 1-5, wherein the ABCB5+ stem cells are dermal mesenchymal stem cells.

7. The method of any one of claims 1-5, wherein the ABCB5+ stem cells are limbal stem cells.

8. The method of any one of claims 1-5, wherein the ABCB5+ stem cells are an isolated population of synthetic ABCB5+ stem cells, wherein greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells.

9. The method of any one of claims 1-8, wherein the ABCB5+ stem cells possess multipotent differentiation capacity.

10. The method of any one of claims 1-8, wherein the ABCB5+ stem cells possess the capacity to differentiate into cells derived from all three germ layers, endoderm, mesoderm and ectoderm.

11. The method of any one of claims 1-8, wherein at least 5% of the ABCB5⁺ stem cells includes an exogenous gene.
